(19) United States
(12) Patent Application Publication
(10) Pub. No.: US 2006/0020121 A1
(43) Pub. Date: Jan. 26, 2006

(54) Preparation of ytterbium(III) beta-diketonates

(76) Inventors: Arne Gerlach, Odenthal (DE); Torsten Groth, Odenthal (DE); Steffen Hofacker, Odenthal (DE)

Correspondence Address: LANXESS CORPORATION, 111 RIDC PARK WEST DRIVE, PITTSBURGH, PA, 15275-1112, US

(21) Appl. No.: 11/182,568

(22) Filed: Jul. 15, 2005

(30) Foreign Application Priority Data
Jul. 20, 2004 (DE) 1020040351295

(51) Int. Cl. C07F 5/00; C07C 51/43

(52) U.S. Cl. 534/15; 554/174

(57) ABSTRACT

The invention relates to a process for preparing ytterbium(III) β-diketonates, especially ytterbium(III) acetylacetonate, to the ytterbium(III) β-diketonates prepared by this process and to the use thereof as catalysts.

The invention relates to a process for preparing ytterbium(III) β-diketonates, especially ytterbium(III) acetylacetonate, to the ytterbium(III) β-diketonates prepared by this process and to the use thereof as catalysts.

Oligocarbonate polyols are important precursors, for example in the production of plastics, coatings and adhesives. To this end, they are reacted, for example, with isocyanates, epoxides, (cyclic) esters, acids or acid anhydrides. Oligocarbonate polyols may in principle be prepared from aliphatic polyols by reacting with phosgene, bischlorocarbonic esters, diaryl carbonates, cyclic carbonates or dialkyl carbonates.

In the case of reaction with alkyl carbonates, for example dimethyl carbonate, transesterification catalysts are frequently used, for example alkali metals or alkaline earth metals and their oxides, alkoxides, carbonates, borates or salts of organic acids (for example WO 2003/2630).

In addition, preference is given to using, as transesterification catalysts, tin or organotin compounds such as bis(tributyltin) oxide, dibutyltin laurate or else dibutyltin oxide (DE-A 2 523 352), and also compounds of titanium, such as titanium tetrabutoxide, titanium tetraisopropoxide or titanium dioxide (for example EP-B 0 343 572, WO 2003/2630).

The transesterification catalysts known from the prior art for the preparation of aliphatic oligocarbonate polyols by reacting alkyl carbonates with aliphatic polyols do, though, have some disadvantages, for example organotin compounds, as potential carcinogens, remaining in subsequent products of the oligocarbonate polyols, additionally required neutralization steps when strong bases such as alkali metals or alkaline earth metals or alkoxides thereof are used as catalysts, or undesired colorations (yellowing) in the course of storage when Ti compounds have been used as catalysts. Titanium catalysts also have the disadvantage of a high activity toward isocyanate-containing compounds in the further reaction of the hydroxyl-terminated oligocarbonates as a polyurethane raw material, so that, to avoid this disadvantage, a further inactivation step for the transesterification catalyst remaining in the product is required. In this inactivation, for example by addition of phosphoric acid (EP-B 1 091 993) or hydrolysis of the titanium compounds (U.S. Pat. No. 4,891,421), an appropriate amount of water is added to the product and has to be removed again from the product by distillation on completion of deactivation. It has also not been possible with the catalysts used to date to lower the reaction temperature, which is typically between 150° C. and 230° C., in order to substantially prevent the formation of by-products such as ethers or vinyl groups, which can form at elevated temperature. These undesired end groups lead, as chain terminators for subsequent polymerization reactions, for example in the case of the polyurethane reaction with polyfunctional (poly)isocyanates, to a lowering of the network density and thus to poorer product properties (for example solvent or acid resistance). In addition, oligocarbonate polyols which have been prepared with the aid of catalysts known from the prior art have high contents of ether groups (for example methyl ethers, hexyl ethers, etc.), which lead in the oligocarbonate polyols, for example, to insufficient hot air stability of cast elastomers, since ether bonds in the material are cleaved under these conditions and thus lead to failure of the material. DE-A 101 56 896 teaches that the disadvantages detailed above can be eliminated by the use of rare earth metal compounds. Possible rare earth metal compounds are ytterbium(III) β-diketonates, in particular ytterbium(III) acetylacetonate.

The literature describes several processes for preparing lanthanide(III) acetylacetonates. For example, J. G. Stites et al. (J. Am. Chem. Soc., 1948, 70, 3142-3143) describe the preparation of lanthanide(III) acetylacetonates from lanthanide(III) chlorides with acetylacetone and ammonia in water. A water-free process for preparing ytterbium(III) acetylacetonate using gaseous ammonia and organic solvents is described in DT-A 25 55 556 A1.

However, when ytterbium(III) acetylacetonate prepared by one of these processes is used, it is found that, when this material is used, for example, as a transesterification catalyst for the reaction of organic carbonate esters with aliphatic polyols, a catalytic activity can be detected, but is unsatisfactory with regard to the space-time yield for commercial preparation of aliphatic oligocarbonates.

It is therefore an object of the present invention to provide rare earth metal compounds, for example ytterbium(III) β-diketonates, which are suitable, for example, as transesterification catalysts and have an increased catalytic activity compared to the prior art. It is a further object of the invention to provide in particular ytterbium(III) acetylacetonate which has an increased catalytic activity compared to the prior art.

It has now been found that, surprisingly, ytterbium(III) β-diketonates which have been prepared from an ytterbium(III) salt, a 1,3-diketone and a base in aqueous solution fulfil this requirement when fewer than 3 equivalents of 1,3-diketone based on 1 equivalent of ytterbium(III) salt are used in the reaction and the resulting ytterbium(III) β-diketonate is dried at at least one temperature of 45° C. to 150° C. for longer than 5 hours.

The present invention therefore provides a process for preparing an ytterbium(III) β-diketonate by reacting at least one ytterbium(III) salt, at least one 1,3-diketone and a base in aqueous solution, characterized in that fewer than 3 equivalents of 1,3-diketone based on 1 equivalent of ytterbium(III) salt are used and the resulting ytterbium(III) β-diketonate is dried at at least one temperature of >50° C. to 150° C. for longer than 5 hours.

In the context of the invention, a base preferably refers to a Brønsted base, the pK_{Λ} value of the acid-base pair in aqueous solution at 25° C. preferably being less than 12. The bases used are preferably ammonia, Na₂CO₃, K₂CO₃ or sodium acetate; particular preference is given to using ammonia, very particular preference to using it in the form of an aqueous solution.

The ytterbium(III) salts used are preferably ytterbium(III) halides or ytterbium(III) nitrate, more preferably ytterbium(III) halides; very particular preference is given to using ytterbium(III) chloride. The ytterbium(III) salts may be used in anhydrous or hydrated form.

The 1,3-diketone used is preferably acetylacetone, dibenzoylmethane or dipivaloylmethane; particular preference is given to using acetylacetone.

The process according to the invention is preferably carried out in such a way that at least one ytterbium(III) salt, preferably one ytterbium(III) salt, and at least one 1,3-diketone, preferably one 1,3-diketone, are initially charged in aqueous solution and an aqueous ammonia solution is added within a suitable time. Ytterbium(III) salt and 1,3-diketone are initially charged preferably at a temperature of 15° C. to 60° C., but preferably anbient temperature. The aqueous ammonia solution is added preferably within a period of 15 minutes to 10 hours, more preferably 30 minutes to 5 hours. In the course of the addition, the temperature rises preferably to temperatures of 15° C. to 60° C. On completion of addition, the reaction mixture is optionally stirred over a further period of 15 min to 10 hours, preferably 30 minutes to 5 hours, and subsequently cooled to a temperature of 1° C. to <15° C., preferably 2° C. to 10° C., more preferably 3° C. to 5° C. The ytterbium(III) β-diketonate is removed from the aqueous mother liquor, for example by filtration, optionally washed once or more than once with water and subsequently dried.

Preference is given to using 2.5 to 2.99 equivalents, more preferably 2.6 to 2.9 equivalents, of 1,3-diketone based on 1 equivalent of ytterbium(III) salt.

Preference is further given to carrying out the reaction of the ytterbium(III) salt with the 1,3-diketone and ammonia in such a way that, after the addition of the ammonia solution, the reaction mixture has a pH of 5 to 10, preferably of 6.5 to 9.5, more preferably of 8 to 9. The pH values relate to the values which are measured at the appropriate temperature of the reaction mixture after addition of the ammonia solution. The measurement is effected generally with a commercial pH electrode (glass electrode).

The drying is effected at at least one temperature of >50° C. to 150° C., preferably of 80 to 130° C., more preferably of 100° C. to 120° C., for more than 5 hours, preferably for 7 to 60 hours, more preferably for 10 to 48 hours.

In a preferred embodiment, the drying is effected at different temperatures, initially at a low temperature which may even be ≦50° C. and subsequently at a higher temperature of >50° C. to 150° C., preferably of 80 to 130° C., more preferably of 100° C. to 120° C. Drying is effected at the low temperature preferably for 1 to 10 hours, more preferably for 3 to 7 hours, and at the higher temperature for more than 5 hours, preferably for 7 to 60 hours, more preferably for 10 to 48 hours.

The drying is also preferably carried out at at least one pressure of ≦1 bar, preferably ≦500 mbar, more preferably ≦100 mbar.

The aqueous ammonia solutions used are preferably in commercially available concentrations. However, it is also possible to use ammonia solutions of any concentration or dilute aqueous ammonia solutions.

In the context of the invention, aqueous solution refers preferably to a solution which comprises water as a solvent. In the context of the invention, such an aqueous solution may also comprise additions of organic solvents. However, an aqueous solution in the context of the invention more preferably comprises substantially water as the solvent.

The ytterbium(III) β-diketonates prepared by the process according to the invention are outstandingly suitable as transesterification catalysts. For example, compared to ytterbium(III) acetylacetonate prepared by known processes, in the transesterification of organic carbonates with aliphatic polyols to prepare aliphatic oligocarbonate polyols, they have a distinctly increased catalytic activity.

The invention therefore further provides ytterbium(III) β-diketonates prepared by the process according to the invention.

Owing to their increased catalytic activity compared to ytterbium(III) β-diketonates prepared by known processes, the ytterbium(III) β-diketonates prepared in accordance with the invention are outstandingly suitable as transesterification catalysts.

The invention therefore further provides for the use of the ytterbium(III) β-diketonates prepared in accordance with the invention as transesterification catalysts.

The examples which follow serve to explain and illustrate the invention by way of example, but do not constitute any restriction.

### EXAMPLES

### Example 1

Preparation of ytterbium(III) acetylacetonate with an acetylacetone/YbCl₃ ratio of 2.8/1.0:
a) A 2000 ml jacketed glass reactor with thermometer and pH electrode was initially charged with 688 g of water, 200 g (0.516 mol, 1.0 equivalent) of ytterbium(m) chloride hexahydrate and 148 g (1.445 mol, 2.8 equivalents) of acetylacetone at room temperature (20° C.). Within one hour, 105 g (1.600 mol, 3.1 equivalents) of a 26% aqueous ammonia solution were added dropwise. During the addition, the temperature rose to 35° C. and the pH to 8.09. The mixture was then stirred for a further hour before it was cooled to 5° C. Filtration and washing with 2×250 ml of water afforded 218 g of crude material. This crude material was subsequently dried at 20 mbar at 50° C. for 5 h and 100° C. for 36 h, and the yield was determined (cf. Tab. 1).
b) Ytterbium(III) acetylacetonate was prepared as described under a) with the alteration that the crude material was dried at 20 mbar at 50° C. for 5 h and at 100° C. for 60 h.

### Comparative Example 1

For comparison, ytterbium(III) acetylacetonate was prepared as described in Example 1 a) with the alteration that the crude material was dried at 20 mbar at 50° C. for 5 h.

On completion of drying, the catalytic activity of the resulting ytterbium(III) acetylacetonate was determined in each case. The results are listed in Tab. 1.

Determination of the Catalytic Activity:

The catalyst activity was determined by preparing an oligocarbonate diol HFC 1947A. As a measure of the activity of the ytterbium(III) acetylacetonate, the hydroxyl number (OHN) was determined for this purpose after preparing the oligocarbonate diol as per DIN 53240-2. Specifically, the following experiment was carried out to determine the activity in each case of the ytterbium(III) acetylacetonate from Example 1 a), b) and Comparative Example 1:

332.1 g of 1,6-hexanediol were dewatered under a nitrogen atmosphere at 120° C. and 20 mbar for 2 h. Afterwards, 352.7 g of dimethyl carbonate and 0.08 g of ytterbium(mi) acetylacetonate were added at 80° C. and reacted under reflux and an inert gas atmosphere for 24 hours. Subsequently, the temperature was increased to 150° C., and methanol and dimethyl carbonate were removed by distillation from the reaction mixture under standard pressure for 4 hours. Afterwards, the temperature was increased to 180° C. and by-products were removed for a further 4 hours. Finally, the temperature was reduced to 130° C. and the pressure to <30 mbar. While simultaneously passing a nitrogen stream (2 1h) through the reaction mixture, further by-products were removed distillatively by continuously increasing the temperature to 180° C. The increase in the temperature from 130 to 180° C. was effected under the temporal proviso that the top temperature did not exceed 60° C. After 180° C. had been attained, this temperature was retained at a pressure of <30 mbar for 4 hours.

Subsequently, the hydroxyl number (OHN) of the resulting oligocarbonate was determined as a measure of the activity of the catalyst to DIN 53240-2. The hydroxyl number refers in this context to that amount of potassium hydroxide in mg which is equivalent to the amount of acetic acid bound in the acetylation of 1 g of substance. The hydroxyl number thus has the unit mg KOH/g. (cf. DIN-53240-2).

The value of the hydroxyl number is thus inversely proportional to the activity of the catalyst used.

TABLE 1

| Catalyst | Temp. | Time | Pressure | Yield | OHN ^{[1]} |
|---|---|---|---|---|---|
| (Yb(acac)₃) | (° C.) | (h) | (mbar) | (g) | (mg KOH/g) |
| Comparative | 50 | 5 | 20 | 203 | 227 |
| Example 1 | | | | | |
| Example 1 a) | 50 | 5 | 20 | 198 | 123 |
| | 100 | 36 | | | |
| Example 1 b) | 50 | 5 | 20 | 196 | 100 |
| | 100 | 60 | | | |

^{[1]} The measured OH number (OHN) is inversely proportional to the activity of the catalyst and is thus a measure of its activity. The OHN and thus indirectly the activity of the ytterbium(III) acetylacetonate were each determined by the performance test described above.

The inventive Examples 1 a) and b) show that, in the course of drying at at least one temperature of >50° C., a considerable rise in activity can be observed in comparison to a catalyst which has been dried merely at 50° C. In addition, Examples 1 a) and b) show that, with increasing drying time, at the elevated temperature, a further distinct increase in activity can be achieved.

### Comparative Example 2

Preparation of ytterbium(III) acetylacetonate with an acetylacetone/YbCl₃ ratio of 3.1/1.0:
a) A 2000 ml jacketed glass reactor with thermometer and pH electrode was initially charged with 688 g of water, 200 g (0.516 mol, 1.0 equivalent) of ytterbium(III) chloride hexahydrate and 163 g (1.600 mol, 3.1 equivalents) of acetylacetone at room temperature (20° C). Within one hour, 105 g (1.600 mol, 3.1 equivalents) of a 26% aqueous ammonia solution were added dropwise. During the addition, the temperature rose to 33° C. and the pH to 8.49. The mixture was then stirred for a further hour before it was cooled to 4° C. Filtration and washing with 2×250 ml of water afforded 319 g of crude material. This crude material was subsequently dried at 20 mbar at 50° C. for 12 h and 100° C. for 24 h, and the yield was determined (Tab. 2).
b) Ytterbium(III) acetylacetonate was prepared as described under a) with the alteration that the crude material was dried at 20 mbar at 50° C. for 12 h and at 100° C. for 48 h.

On completion of drying, the catalytic activity of the resulting ytterbium(III) acetylacetonate was in each case determined as described under “determination of the catalytic activity”. The results are listed in Table 2.

TABLE 2

| Catalyst | Temp. | Time | Pressure | Yield | OHN ^{[1]} |
|---|---|---|---|---|---|
| (Yb(acac)₃) | (° C.) | (h) | (mbar) | (g) | (mg KOH/g) |
| Comparative | 50 | 12 | 20 | 212 | 145 |
| Example 2 a) | 100 | 24 | | | |
| Comparative | 50 | 12 | 20 | 208 | 153 |
| Example 2 b) | 100 | 48 | | | |

^{[1]} cf. Tab.1

Comparative Examples 2a) and b) show that, when more than 3 equivalents of acetylacetone based on 1 equivalent of YbCl₃ are used, even with drying temperatures of >50° C., no increase in activity such as that in the case of the inventive catalysts from Examples 1a) and b) can be observed. In addition, Comparative Examples 2a) and b) show that, even with prolonged drying time, no significant increase in activity was achieved here.

1. Process for preparing an ytterbium(III) β-diketonate by reacting at least one ytterbium(El) salt, at least one 1,3-diketone and a base in aqueous solution, characterized in that fewer than 3 equivalents of 1,3-diketone based on 1 equivalent of ytterbium(III) salt are used and the resulting ytterbium(III) β-diketonate is dried at at least one temperature of >50° C. to 150° C. for longer than 5 hours.

2. Process according to claim 1, characterized in that the ytterbium(III) salt used is ytterbium(III) chloride.

3. Process according to claim 1, characterized in that the 1,3-diketone used is acetylacetone.

4. Process according to claim 1, characterized in that the base used is ammonia.

5. Process according to claim 1, characterized in that the drying time is 10 to 48 h.

6. Process according to claim 1, characterized in that 2.5 to 2.99 equivalents of 1,3-diketone based on 1 equivalent of ytterbium(III) salt are used.

7. Process according to claim 1, characterized in that the reaction is carried out at at least one temperature of 15° C. to 60° C.

8. Process according to claim 1, characterized in that the drying is carried out at at least one pressure of ≦1 bar.

9. Ytterbium(III) β-diketonate prepared by a process according to claim 1.

10. Transesterifikation process comprising using ytterbium(III) β-diketonate according to claim 9 as a transesterification catalyst.
